(19) United States
(12) Patent Application Publication
(10) Pub. No.: US 2019/0048359 A1
(43) Pub. Date: Feb. 14, 2019

(54) NOVEL BIOGASOLINE PRODUCTION VIA MACROALGAL DUCKWEEDS BY THE OPERON EXPRESSION METHOD IL-60

(71) Applicants: NICOLAS LAQUA, SAN DIEGO, CA (US); ANNAM TRAN, DEL MAR, CA (US)

(72) Inventors: NICOLAS LAQUA, SAN DIEGO, CA (US); ANNAM TRAN, DEL MAR, CA (US)

(21) Appl. No.: 16/101,434

(22) Filed: Aug. 11, 2018

Related U.S. Application Data
(60) Provisional application No. 62/544,817, filed on Aug. 12, 2017.

(51) Int. Cl. C12N 15/82

(52) U.S. Cl. CPC C12N 15/8243; C12N 2750/12043; C12N 15/8218

(57) ABSTRACT

The present disclosure describes methods of viral induced gene expression in aquatic duckweeds such as Azolla caroliniana and Lemna minor for the production of acetone, butanol, and ethanol for biofuel production.

### CROSS REFERENCE TO RELATED APPLICATIONS

This application claims benefit of U.S. Provisional Patent Application No. 62/544,817, filed Aug. 12, 2017. The prior application is incorporated by reference herein in its entirety.

### INCORPORATION BY REFERENCE

All publications, patents, patent applications, public databases, public database entries, and other references cited in this application are herein incorporated by reference in their entirety as if each individual publication, patent, patent application, public database, public database entry, or other reference was specifically and individually indicated to be incorporated by reference.

### REFERENCES

Nair, Ramesh V. et al. “Regulation of the sol Locus Genes for Butanol and Acetone Formation in Clostridium Acetobutylicum ATCC 824 by a Putative Transcriptional Repressor.” Journal of Bacteriology 181.1 (1999): 319-330. Nölling, Jörk et al. “Genome Sequence and Comparative Analysis of the Solvent-Producing Bacterium Clostridium Acetobutylicum.” Journal of Bacteriology 183.16 (2001): 4823-4838. PMC. Web. 13 Feb. 2017. Monot, Frédéric et al. “Acetone and Butanol Production by Clostridium Acetobutylicum in a Synthetic Medium.” Applied and Environmental Microbiology 44.6 (1982): 1318-1324. Yang, Shang-Tian, Ohio State University; “A Novel Fermentation Process for Butyric Acid and Butanol Production from Plant Biomass,” The Consortium for Plant Biotechnology Research, Inc., Environmental Research and Technology Transfer Program (2001), Online Database for EPA Research Papers. Arora, A., and P. K. Singh, (2003), “Comparison of biomass productivity and nitrogen fixing potential of Azolla SPP, Biomass and Bioenergy” National Centre for Conservation and Utilization of Blue Green Algae, Indian Agricultural Research Institute, New Delhi 110012, India. Cary, P. R. and P. G. J. Weerts (1991) “Growth and nutrient composition of Azolla pinnata R. Brown and Azolla filiculoides Lamarck as affected by water temperature, nitrogen and phosphorus supply, light intensity and pH.” Aquat. Bot 43. Nakayama, Shunichi et al. “Butanol Production from Crystalline Cellulose by Cocultured Clostridium Thermocellum and Clostridium Saccharoperbutylacetonicum N1-4.” Applied and Environmental Microbiology 77.18 (2011): 6470-6475. PMC. Web. 13 Feb. 2017. Peretz, Yuval et al. “A Universal Expression/Silencing Vector in Plants.” Plant Physiology 145.4 (2007): 1251-1263. PMC. Web. 13 Feb. 2017. Gover, Ofer et al. “Only Minimal Regions of Tomato Yellow Leaf Curl Virus (TYLCV) Are Required for Replication, Expression and Movement.” Archives of Virology 159.9 (2014): 2263-2274. PMC. Web. 13 Feb. 2017. Anderson D G, McKay L L. Simple and rapid method for isolating large plasmid DNA from lactic streptococci. Applied and Environmental Microbiology. 1983; 46(3):549-552. Roberts, I, W M Holmes, and P B Hylemon. “Modified Plasmid Isolation Method for Clostridium Perfringens and Clostridium Absonum.” Applied and Environmental Microbiology 52.1 (1986): 197-199.

### BACKGROUND

Due to the unparalleled ease of compression, storage, and transport of long chain hydrocarbon sources such as gasoline, biofuel production is a crowded area of research. Most biofuel research focuses on either microorganisms, such as microalgae, or very large crops, namely cereal crops such as corn or seed crops such as sunflower, mustard, or rapeseed/canola. However, problems arise with both strategies: the use of cereal crops for fuel crop farming produces very little oil per gallon/acre and consumes valuable agricultural land, often in areas with an agrarian economy, while microalgae, though generally several orders of magnitude more productive than the former, due to the rapid, exponential growth exhibited by the crop, has faced barriers relating to reliable harvest that have prevented it from actualizing as a usable biofuel—as flocculation and then transesterification are generally required to process the microalgal-derived biofuel, as opposed to merely just transesterification. Because current biofuel production methods, although renewable, are not sustainable, alternative, cost effective methods of using aquatic duckweeds to produce biofuels need to be developed.

### SUMMARY

Provided herein are modified macroalga duckweeds useful for the production of acetone, butanol, or ethanol. In some embodiments, the macroalga duckweed is Azolla caroliniana or Lemna minor. In other embodiments, the modification is the inoculation of the macroalga duckweed with a viral-based construct, for example, IL-60-BS. In one embodiment, the inoculation is via root uptake in the macroalga duckweed. In other embodiments, the modified macroalga duckweed has an increase in dry weight acetone content (%/g), dry weight butanol content (%/g), or dry weight ethanol content (%/g), as compared to an unmodified (control) macroalga duckweed of the same species, or over a period of time, for example, over one or more generations. In some embodiments, the increase in dry weight butanol content (%/g), is 0.01% to 0.5%, 0.5% to 1%, 1% to 1.5%, 1.5% to 2%, 2% to 2.5%, 2.5% to 3%, 3% to 3.5%, or 3.5% or more.

Disclosed herein are modified macroalga duckweeds useful for production of acetone, butanol, or ethanol. In some embodiments, the duckweed is Azolla caroliniana or Lemna minor. In one embodiment, the modification is inoculation with a viral-based construct. In another embodiment, the viral-based construct is IL-60-BlueScript (BS). In one embodiment, the inoculation is via root uptake in the macroalga duckweed. In another embodiment, the viral-based construct comprises a 60-base-pair deletion inhibiting rolling circle replication. In another embodiment, the viral-based construct comprises ctfA, ctfB, adc, and aad. In other embodiments, the modified macroalga duckweed has an increase in dry weight acetone content (%/g), dry weight butanol content (%/g), or dry weight ethanol content (%/g), as compared to an unmodified (control) macroalga duckweed of the same species. In other embodiments, the increase in dry weight acetone content (%/g), dry weight butanol content (%/g), or dry weight ethanol content (%/g), is 0.01% to 0.5%, 0.5% to 1%, 1% to 1.5%, 1.5% to 2%, 2% to 2.5%, 2.5% to 3%, 3% to 3.5%, or 3.5% or more. In other embodiments, the modified macroalga duckweed has an increase in dry weight acetone content (%/g), dry weight butanol content (%/g), or dry weight ethanol content (%/g), as compared to an unmodified (control) macroalga duckweed of the same species over a period of time. In one embodiment, the period of time is a generation or more. In one embodiment, the modified macroalga duckweed has a similar growth rate as compared to an unmodified (control) macroalga duckweed of the same species over a period of time. In one embodiment, the period of time is a generation or more.

Also disclosed is a modified macroalga duckweed wherein the viral-based construct comprises a disarmed tomato yellow leaf curl virus (TYLCV) and a 210 kb plasmid (pSOL1) obtained from Clostridium acetobutylicum ATCC 824 comprising ctfA, ctfB, adc, and aad.

Also disclosed is a modified macroalga duckweed of claim 3, wherein IL-60-BS comprises a disarmed tomato yellow leaf curl virus (TYLCV) and a SolR/SalI operon of pSOL1.

Also disclosed is a modified Azolla caroliniana wherein the modification is the induction of the Azolla caroliniana with a viral-based vector comprising a disarmed tomato yellow leaf curl virus (TYLCV) and a 210 kb plasmid (pSOL1) obtained from Clostridium acetobutylicum ATCC 824 comprising ctfA, ctfB, adc, and aad, wherein the induction is via root uptake and wherein ctfA, ctfB, adc, and aad are expressed and expression results in increased production of acetone or butanol as compared to an unmodified Azolla caroliniana.

Also disclosed is a modified Lemna minor wherein the modification is the induction of the Lemna minor with a viral-based vector comprising a disarmed tomato yellow leaf curl virus (TYLCV) and a 210 kb plasmid (pSOL1) obtained from Clostridium acetobutylicum ATCC 824 comprising ctfA, ctfB, adc, and aad, wherein the induction is via root uptake and wherein ctfA, ctfB, adc, and aad are expressed and expression results in increased production of acetone or butanol as compared to an unmodified Lemna minor.

### BRIEF DESCRIPTION OF THE DRAWINGS

These and other features, aspects, and advantages of the present disclosure will become better understood with regard to the following description, appended claims and accompanying figures.

FIG. 1 shows dry weight percentage of butanol content in a modified strain of A. caroliniana.

FIG. 2 shows a A. caroliniana control strain versus a modified strain AA.

FIG. 3 shows a A. caroliniana control strain versus a modified strain AB.

FIG. 4 shows a A. caroliniana control strain versus a modified strain AC.

FIG. 5 shows dry weight percentage of butanol content in a modified strain of L. minor.

FIG. 6 shows butanol dry weight percentage of a L. minor control strain versus a modified strain DA.

FIG. 7 shows butanol dry weight percentage of a L. minor control strain versus a modified strain DB.

FIG. 8 shows butanol dry weight percentage of a L. minor control strain versus a modified strain DC.

FIG. 9 shows butanol dry weight percentage of a L. minor control strain versus a modified strain DD.

FIG. 10 shows butanol dry weight percentage of a L. minor control strain versus a modified strain DE.

FIG. 11 is an experimental overview.

FIG. 12 illustrates Soxhlet extraction of Azolla caroliniana and Lemna minor.

FIG. 13 is a photograph of trypticase soy agar plates used in the disclosed methods.

FIG. 14 is a photograph of trypticase soy agar plates used in the disclosed methods.

FIG. 15 is a photograph of a Lemna minor strain sample in an inoculation solution.

FIG. 16 is a map of the 210-kb pSOL1 plasmid of C. acetobutylicum ATCC 824.

FIG. 17 shows chemical structures of various isomers of butanol.

FIG. 18 shows butanol content a modified C. acetobutylicum versus a control C. acetobutylicum.

FIG. 19 shows butanol content a modified L. minor versus a control L. minor.

FIG. 20 shows steps for making IL-60-BS pBlueScript.

### DETAILED DESCRIPTION

The following detailed description is provided to aid those skilled in the art in practicing the present disclosure. Even so, this detailed description should not be construed to unduly limit the present disclosure as modifications and variations in the embodiments discussed herein can be made by those of ordinary skill in the art without departing from the spirit or scope of the present disclosure.

As used in this disclosure and the appended claims, the singular forms “a”, “an” and “the” include a plural reference unless the context clearly dictates otherwise. As used in this disclosure and the appended claims, the term “or” can be singular or inclusive. For example, A or B, can be A and B.

About

The term “about” generally refers to plus or minus 10% of the indicated number. For example, “about 10%” may indicate a range of 9% to 11%, and “about I” may mean from 0.9-1.1.

Ranges

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

Host Cells or Host Organisms

Compounds, such as acetone, ethanol, butanol and other naturally produced chemicals, can be obtained from host cells or host organisms. A host organism can be, for example, a macroalga, a macroalga duckweed, Azolla caroliniana, or Lemna minor.

Macroalgae

A host cell or organism can be a macroalga. Macroalgae are aquatic plants that grow upon the surface of water columns.

Macroalgal Ferns

A host cell or organism can be a macroalgal fern. Macroalgal ferns are macroalgae that are classified within the class Polypodiopsida.

Macroalgal Duckweeds

The macroalgal duckweeds Azolla caroliniana and Lemna minor were selected for several reasons, including significantly reduced difficulty of obtaining a reliable harvest as compared to microalgae, as well as the rapid growth employed by both Azolla caroliniana and Lemna minor. A. caroliniana achieved a doubling of biomass every 6.1 days, with a “normal photoperiod, as described in Arora, A., and P. K. Singh, (2003), “Comparison of biomass productivity and nitrogen fixing potential of Azolla SPP, Biomass and Bioenergy” National Centre for Conservation and Utilization of Blue Green Algae, Indian Agricultural Research Institute, New Delhi 110012, India). In addition, Azolla caroliniana has a doubling (of biomass) time of roughly 5.3 days when exposed to constant light. In contrast, L. minor is reported to have a doubling time of just 0.66 generations per day, which corresponds to a doubling time of 16-24 hours, as described in DWRP, 1997 (Große, Wolfgang. “Pressurized ventilation in floating-leaved aquatic macrophytes.” Aquatic Botany 54.2-3 (1996): 137-50). The rate in which L. minor doubles, depends on photoperiod, temperature, and the nutrient content of the water that it is grown in.

Reproduction of Lemna minor is primarily asexual, with one frond producing ten to twenty identical fronds during its several week-long lifetime. Similarly, A. caroliniana reproduces primarily asexually as well, with parent fronds budding frequently; although sexual reproduction from spores is prevalent, as compared to L. minor's nearly exclusive asexual reproduction.

To increase butanol production in both Azolla caroliniana and Lemna minor, nontransgenic IL-60 based expression of operons found within the pSol1 plasmid was performed. Upon expression of the operons, an increase in butanol production in the rapidly growing Azolla caroliniana and Lemna minor was obtained at a rate deemed “percent efficiency”, referring to production efficiency, or dry weight percentage of butanol usable in the production of biogasoline.

After modification, both modified strains had increased butanol content as compared to a control group of the same strain that was not modified. Azolla caroliniana with an elevation of almost ˜2% during its initial parent generation, and an overall 1% increase over multiple generations despite less and less traces of operon being detected. Lemna minor showed a significant ˜3.3% elevation of butanol levels during its initial parent generation while levelling off at an overall 1.2% increase over multiple generations. Based upon the experiments described herein, A. caroliniana, and to a greater. L. minor, are confirmed to be useful organisms for elevated butanol production, which serves as a backbone for continued research into renewable, reliable, carbon negative biogasoline production from fast growing macroalgal sources.

Lemna minor and Azolla caroliniana are remarkably fast growing duckweeds, or macroalgae that grows at or on the surface of standing or slow moving water. Both can double their biomass in under seven days, and are among the fastest growing vascular plants in existence. As such, Lemna minor and Azolla caroliniana were chosen as organisms for the elevation of biogasoline production via the IL-60 based expression of operons found within the pSol1 plasmid of Clostridium acetobutylicum (ATCC 824). Azolla sp. and Lemna serve as useful model organisms that demonstrate expression retention of butanol-elevating properties, as demonstrated via the continued transgenerational elevation of butanol when compared to unmodified specimens, after asexual reproduction.

Elevation of biogasoline could lead to a renewable supply of readily available renewable biofuel. Duckweeds, in combination with the versatile nature of IL-60, can act as model organisms for transgenerational effects of IL-60 based operon expression.

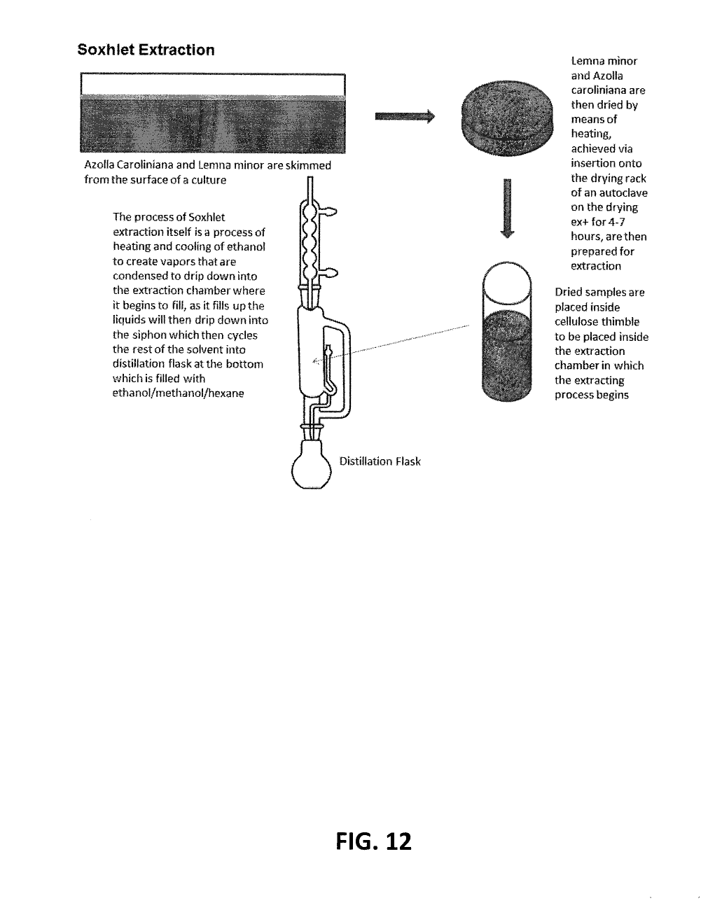

Lemna minor and Azolla caroliniana grow best in industrial wastewater, or phosphorous and nitrogen rich runoff from farmland. Furthermore, extraction, typically one of the most difficult steps in processing alternative biofuels is simple in macroalgal duckweeds. Due to the large size of macroalgal duckweeds they are easy to harvest. A pond skimmer or filter can be used to harvest the duckweed with minimal energy input. Then, a soxhlet extractor or heat press easily extracts the butanol (for example, as shown in FIG. 12). In addition, the leftover organic material may be utilized for fertilization, feed, or as a bio-compost. These environmentally conducive, usable byproducts are valuable assets discovered through the disclosed methods. After extraction, the elevated butanol levels found in the modified strains of A. caroliniana and L. minor resulted in carbon-negative, easily producible biogasoline that only requires sunlight and small amounts of standing water, for example, contaminated runoff from farms, for growth.

Disclosed herein are methods to elevate naturally occurring butanol levels. Also disclosed are organisms useful in the described methods. IL60 based expression of the pSol1 plasmid of Clostridium in two species of duckweed significantly elevated the levels of butanol. An IL-60-BS construct was made via disarmament of the tomato yellow leaf curl virus that contains operons from a PSol1 plasmid that is isolated from Clostridium into the aforementioned construct; then the duckweeds were induced with the virally-based construct via root uptake, resulting in increased expression levels of butanol via introduction of the Clostridium ace. operons in macroalgae, which was ultimately successful and elevated butanol dry weight percentage by as much as 3% in select organisms while modeling the active transgenerational degradation of expression that occurs after asexual reproduction. The described results prove fundamentally that operon expression can increase total biofuel production and operate transgenerationally in a manner that shows complete decadence by the F3 generation.

L. minor and A. caroliniana require nothing more than several centimeters of water and not only exist, but thrive in industrial wastewater (especially nitrogen-rich runoff water contaminated with excessive amounts of fertilizer). Growing on the surface of slow-moving or standing water, azolla and duckweed are valuable in their productivity and scalability, while, due to their short lifespans, serve as model organisms by which projection of behavior may be applied to larger, conventional biofuel producing plants.

Vascular Plants

In other embodiments, the host organism is a vascular plant. Non-limiting examples of such plants that can be used to produce compounds useful in biofuel production include various monocots and dicots, including high oil seed plants such as high oil seed Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annuus), flax (Linum usitatissimum), corn (Zea mays), coconut (Cocos nucifera), palm (Elaeis guineensis), oil nut trees such as olive (Olea europaea), sesame, and peanut (Arachis hypogaea), as well as Arabidopsis, tobacco, wheat, barley, oats, amaranth, potato, rice, tomato, and legumes (e.g., peas, beans, lentils, alfalfa, etc.).

A plant can be, for example, Arabidopsis thaliana, or a Brassica, Glycine, Gossypium, Medicago, Zea, Sorghum, Oryza, Triticum, or Panicum species.

IL-60

As described in Gover, O., et al. (Archives in Virology, April 2014, DOI 10.1007/s00705-014-2066-7), tomato yellow leaf curl virus (TYLCV) is a monopartite begomovirus with a characterized genome organization consisting of six overlapping transcribed open reading frames (ORFs). The six overlapping open reading frames (ORFs) are transcribed in opposite directions from two promoters situated at either end of an intergenic region (IR). The 314-bp IR carries the universal motif TAATATT/AC. In addition to carrying promoters, it also serves as the viral origin of replication. It carries motifs (iterons) for binding the replicase-associated protein (REP—the product of ORF C1). Two ORFs are expressed in viral (sense) orientation—V2 and V1 (pre-coat and coat protein (CP), respectively)—and four ORFs in the complementary orientation (C1 to C4).

The IL-60 platform consists of a disarmed form of tomato yellow leaf curl virus (TYLCV) and auxiliary components, and was developed as a nontransgenic universal vector system for gene expression and silencing that can express an entire operon in plants. IL-60 does not allow rolling-circle replication; hence, production of viral single-stranded (ss) DNA progeny is prevented. This double-stranded (ds) DNA-restricted platform (uncoupled from the dsDNA→ssDNA replication phase of progeny viral DNA) can be used for functional genomics studies of TYLCV.

Clostridium Acetobutylicum ATCC 824

The genome sequence of the solvent-producing bacterium Clostridium acetobutylicum ATCC 824 was determined by the shotgun approach. The genome consists of a 3.94-Mb chromosome and a 192-kb megaplasmid that contains the majority of genes responsible for solvent production. Comparison of C. acetobutylicum to Bacillus subtilis reveals significant local conservation of gene order, which has not been seen in comparisons of other genomes with similar, or, in some cases closer, phylogenetic proximity. This conservation allows the prediction of many previously undetected operons in both bacteria. However, the C. acetobutylicum genome also contains a significant number of predicted operons that are shared with distantly related bacteria and archaea but not with B. subtilis. Phylogenetic analysis is compatible with the dissemination of such operons by horizontal transfer. The enzymes of the solventogenesis pathway and of the cellulosome of C. acetobutylicum comprise a new set of metabolic capacities not previously represented in the collection of complete genomes. These enzymes show a complex pattern of evolutionary affinities, emphasizing the role of lateral gene exchange in the evolution of the unique metabolic profile of the bacterium. Many of the sporulation genes identified in B. subtilis are missing in C. acetobutylicum, which suggests major differences in the sporulation process. Thus, comparative analysis reveals both significant conservation of the genome organization and pronounced differences in many systems that reflect unique adaptive strategies of the two gram-positive bacteria. See Nolling, J., et al., J Bacteriol. 2001 August; 183(16):4823-38, incorporated by reference herein in its entirety.

As described in Cornillot, E., et al., Journal of Bacteriology (1997) Vol. 179, No. 17, p. 5442-5448, incorporated by reference in its entirety, strain ATCC 824 contains a 210-kb plasmid (pSOL1). pSOL1 carries the four acetone and butanol formation genes ctfA, ctfB, adc, and aad.

Compounds Made by the Organisms of the Disclosure

Several compounds can be made using viral induced gene expression in macroalgae, such as aquatic duckweeds. Acetone, butanol, and ethanol can all be produced and used for biofuel production.

Butanol

Butanol (also called butyl alcohol) is a four-carbon alcohol with a formula of C₄H₉OH, which occurs in five isomeric structures, from a straight-chain primary alcohol to a branched-chain tertiary alcohol; all are a butyl or isobutyl group linked to a hydroxyl group (sometimes represented as BuOH, n-BuOH, and i-BuOH). These are n-butanol, 2 stereoisomers of 2-butanol, tert-butanol, and isobutanol. Butanol is primarily used as a solvent, as an intermediate in chemical synthesis, and as a fuel. It is sometimes also called biobutanol when produced biologically.

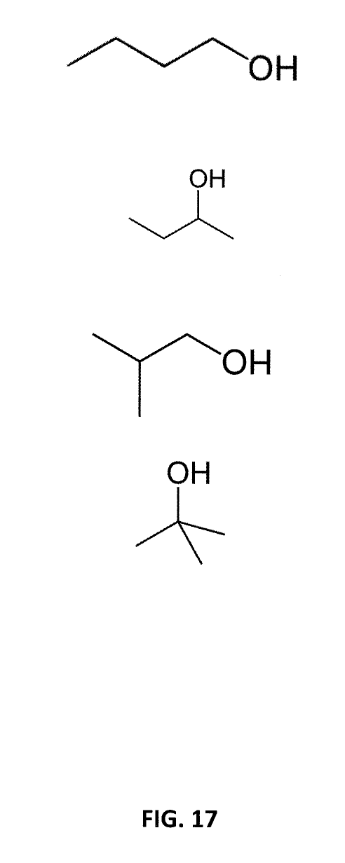

The unmodified term butanol usually refers to the straight chain isomer with the alcohol functional group at the terminal carbon, which is also known as n-butanol or 1-butanol. The straight chain isomer with the alcohol at an internal carbon is sec-butanol or 2-butanol. The branched isomer with the alcohol at a terminal carbon is isobutanol or 2-methyl-1-propanol, and the branched isomer with the alcohol at the internal carbon is tert-butanol or 2-methyl-2-propanol. The structure of n-butanol (1-butanol), sec-butanol, isobutanol, and tert-butanol is shown in FIG. 17 (top to bottom of page).

1-Butanol, which is also known as n-butanol or 1-butanol or butyl alcohol (sometimes also called biobutanol when produced biologically), is an alcohol with a 4 carbon structure and the molecular formula of C₄H₁₀O or CH₃(CH₂)₃OH.

Butanol is primarily used as a solvent, as an intermediate in chemical synthesis, and as a fuel. There are four isomeric structures for butanol. The straight chain isomer with the alcohol at an internal carbon is sec-butanol or 2-butanol. The branched isomer with the alcohol at a terminal carbon is isobutanol, and the branched isomer with the alcohol at the internal carbon is tert-butanol.

Acetone

Acetone ((CH3)2CO), an organic compound, is the simplest ketone.

Ethanol

Ethanol (C2H6O), or ethyl alcohol, is a common alcohol that is produced via both petrochemical processes and fermentation.

Vectors

pBluescript can be purchased from any supplier, for example, Agilent Technologies (USA).

pDrive can be purchased from any supplier, for example, from Qiagen (USA).

Tomato Yellow Leaf Curl Virus was obtained from The University of California, Davis.

A “viral-based” vector is a virus that has been disarmed such that rolling circle replication cannot replicate as efficiently as a virus that has not been disarmed.

The term “vector” and “construct” is interchangeable.

### EXAMPLES

The following examples are intended to provide illustrations of the application of the present disclosure. The following examples are not intended to completely define or otherwise limit the scope of the disclosure.

One of skill in the art will appreciate that many other methods known in the art may be substituted in lieu of the ones specifically described or referenced herein.

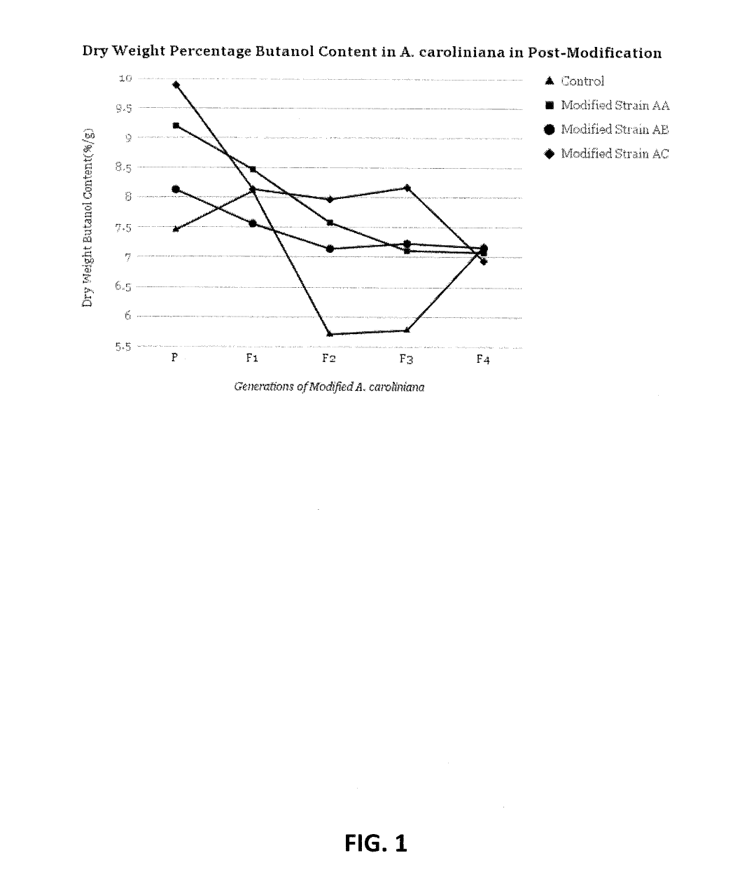

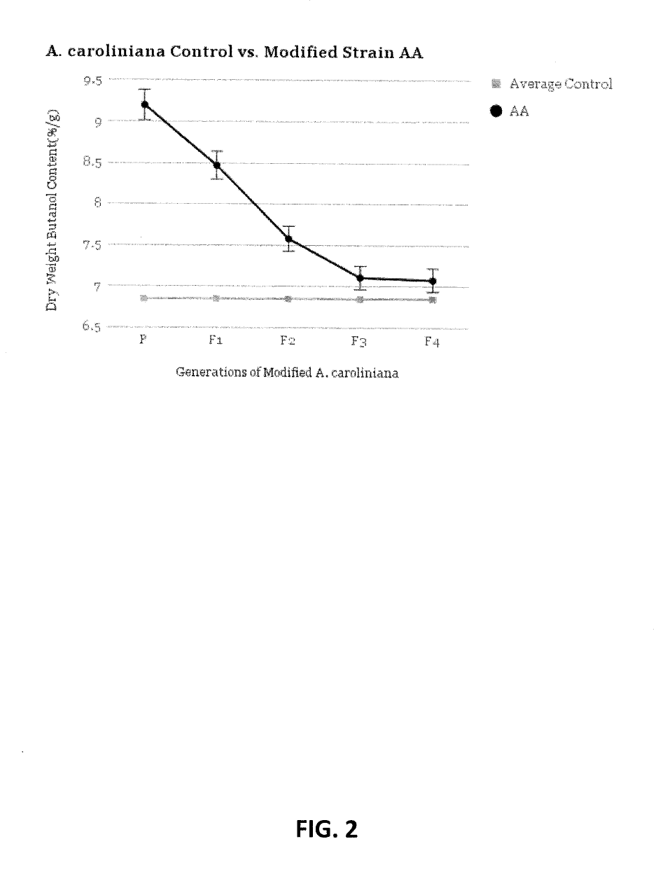

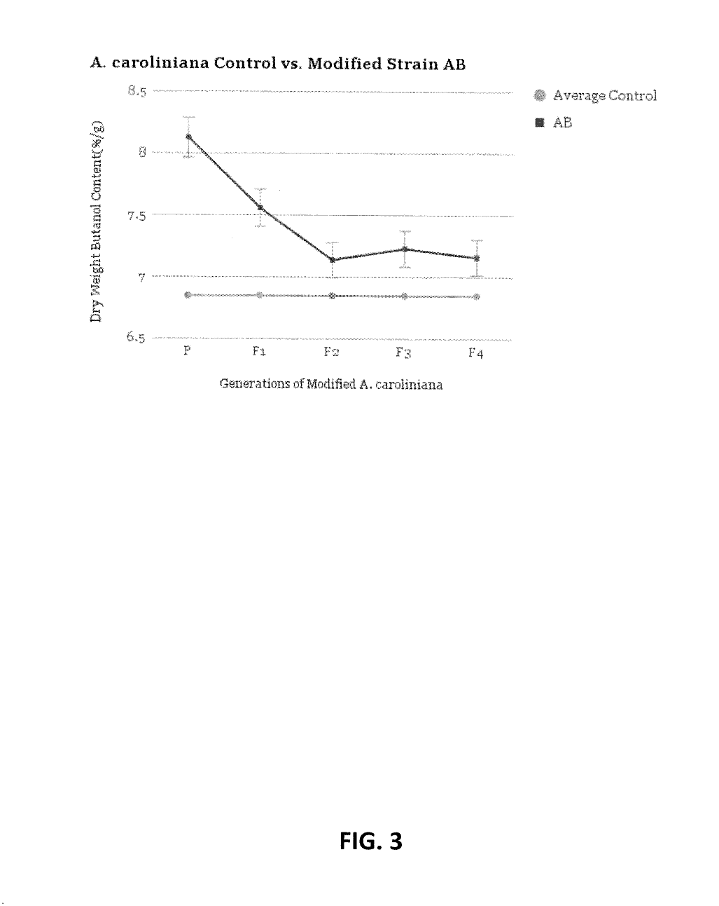

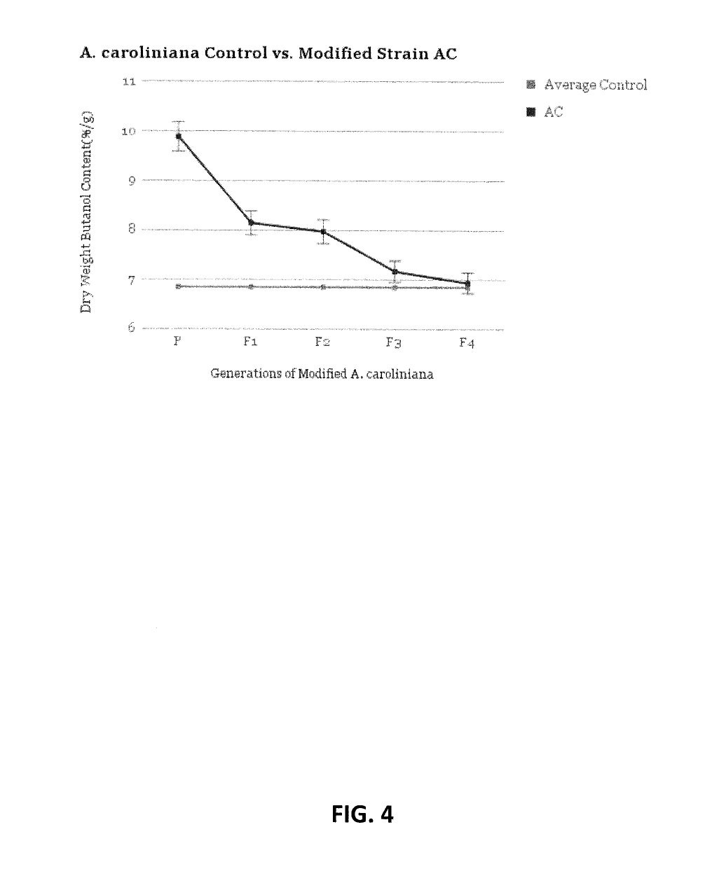

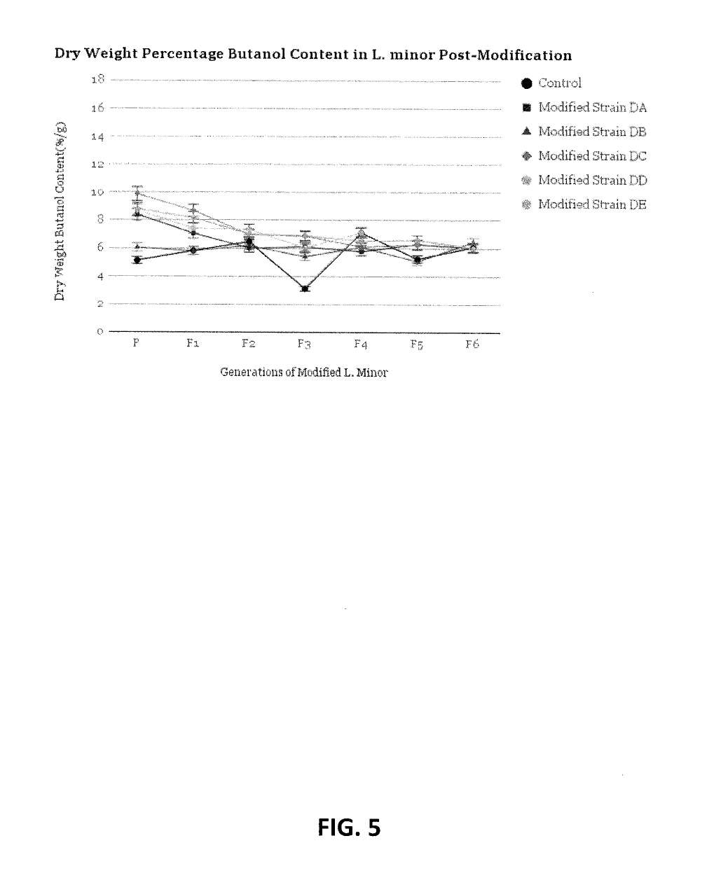

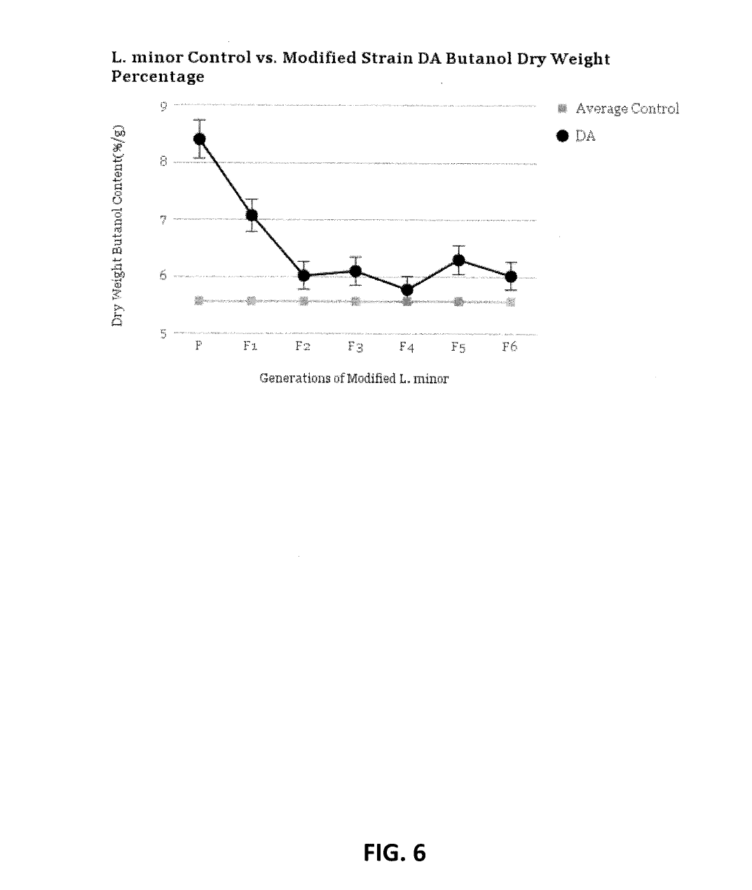

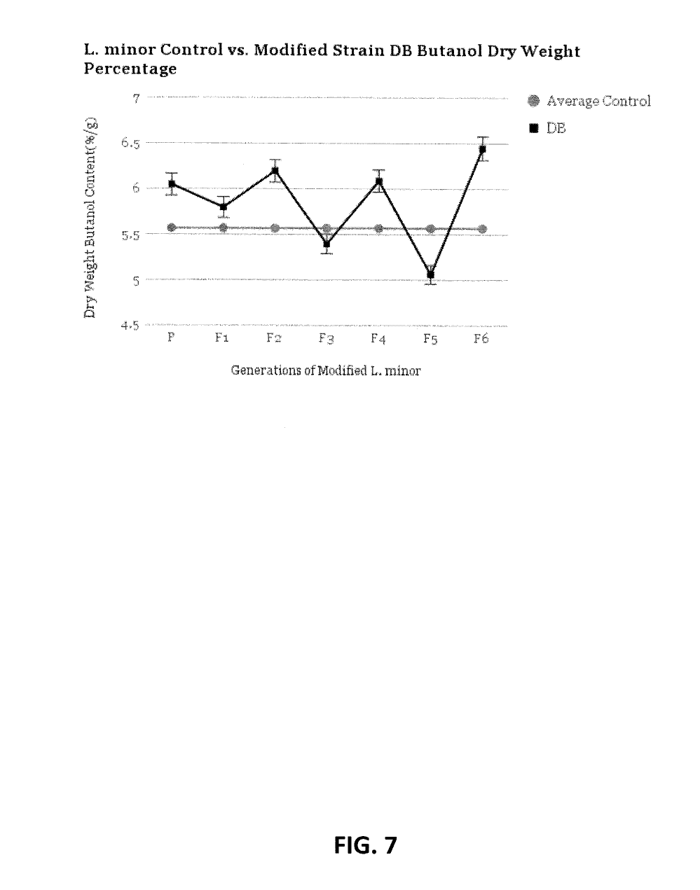

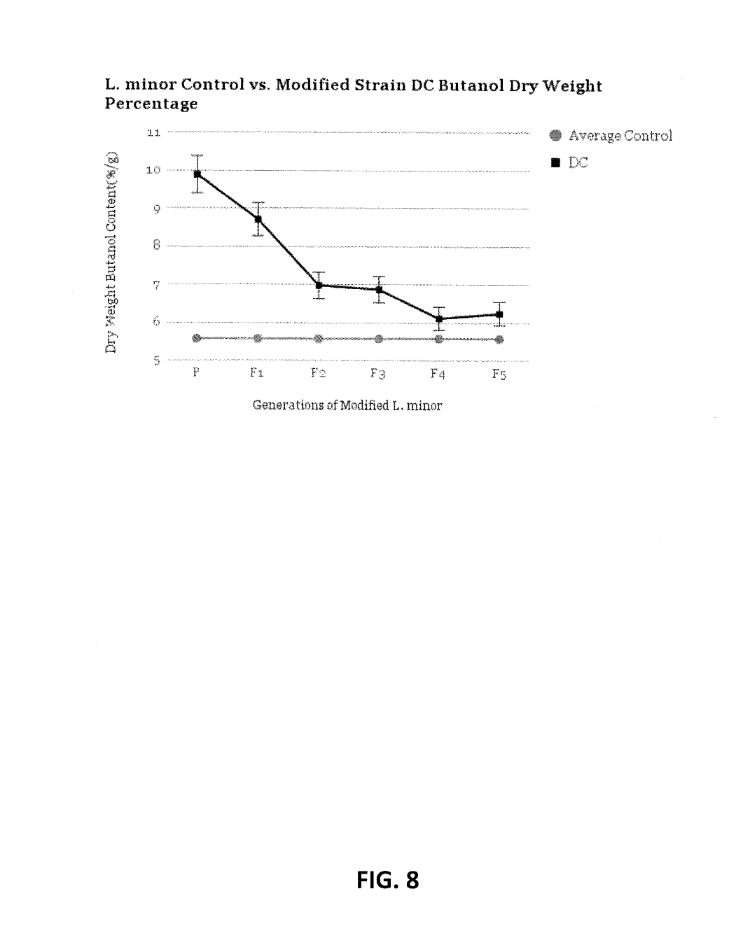

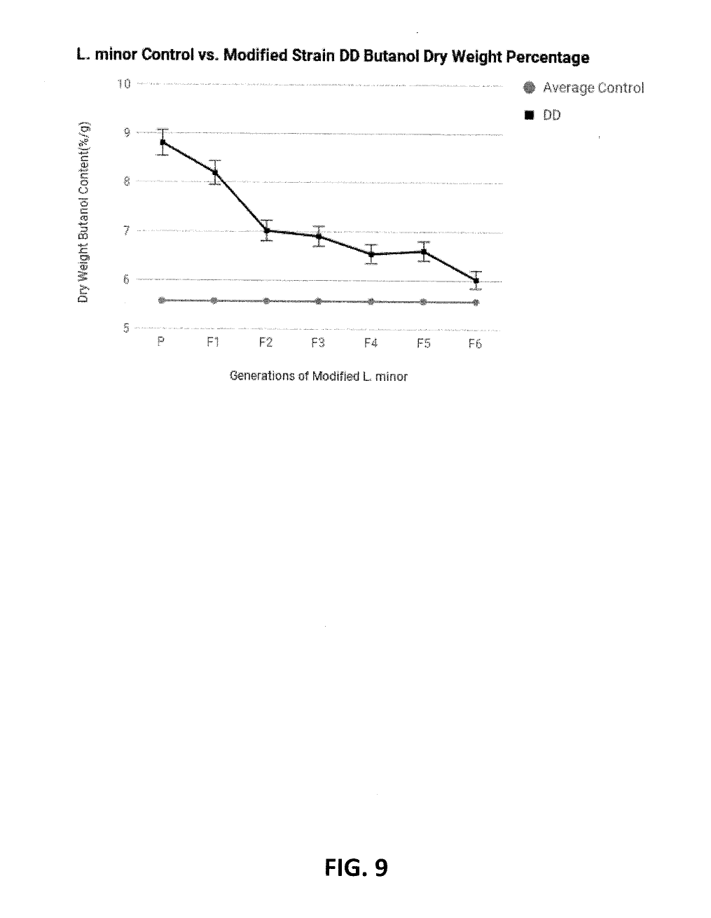

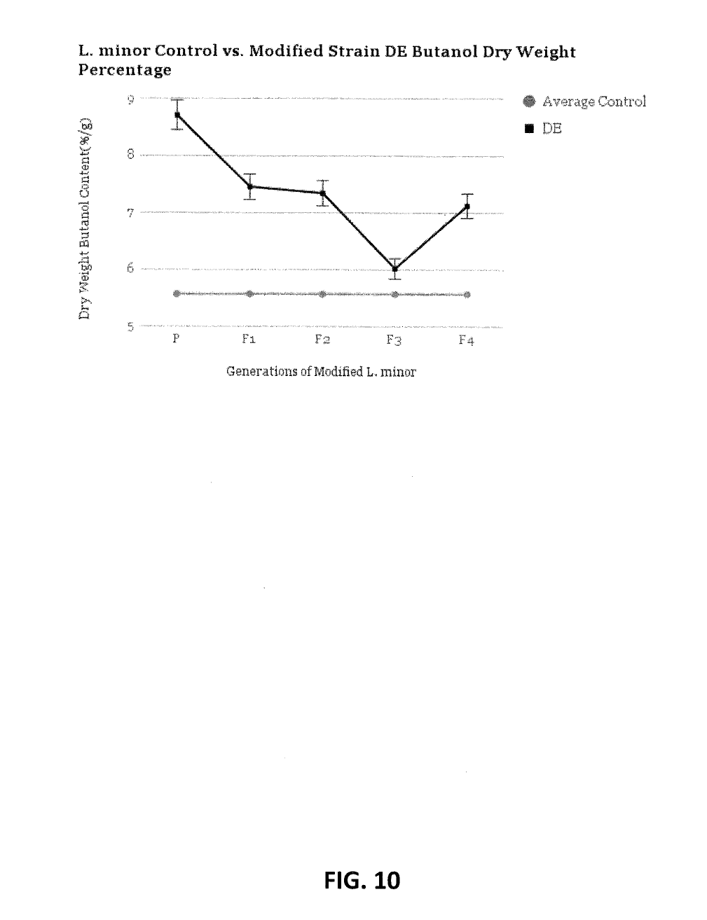

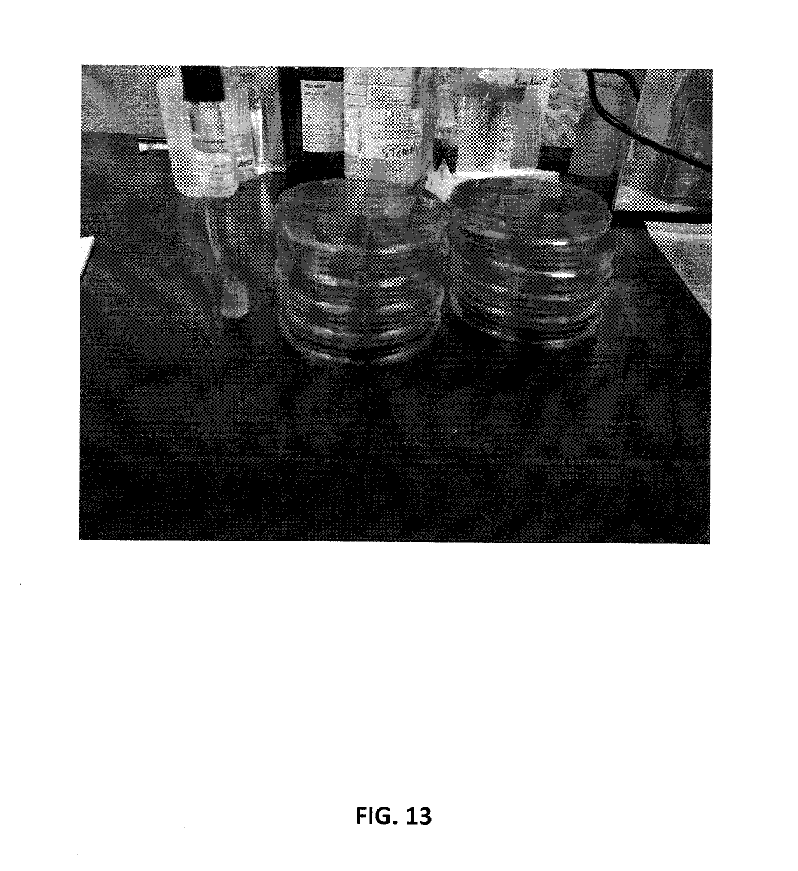

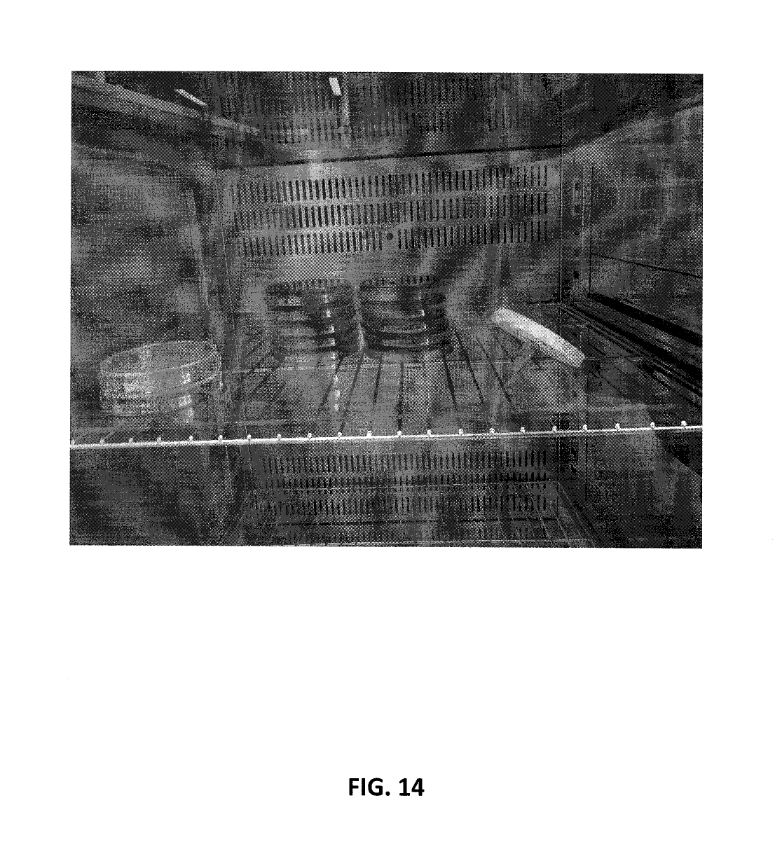

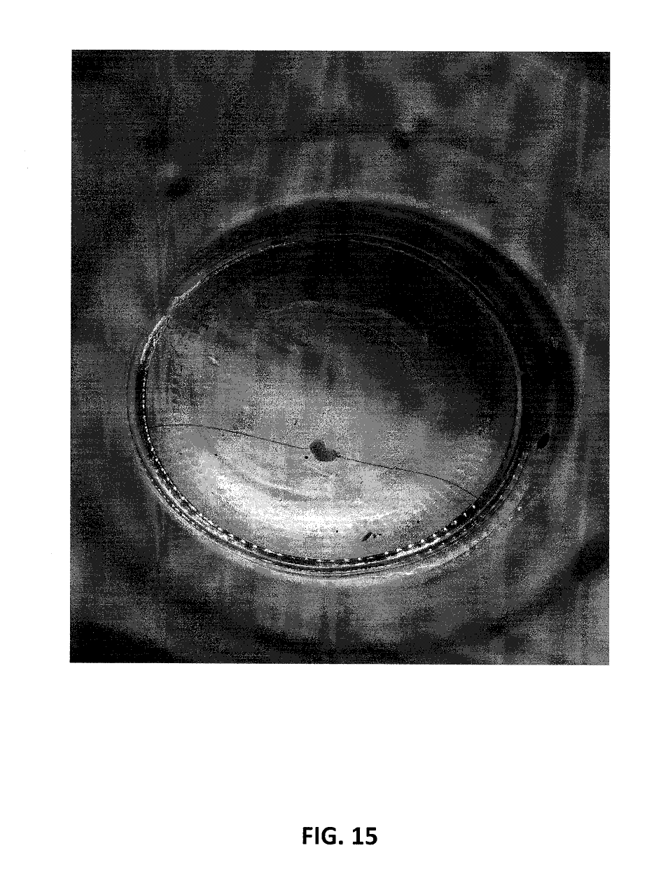

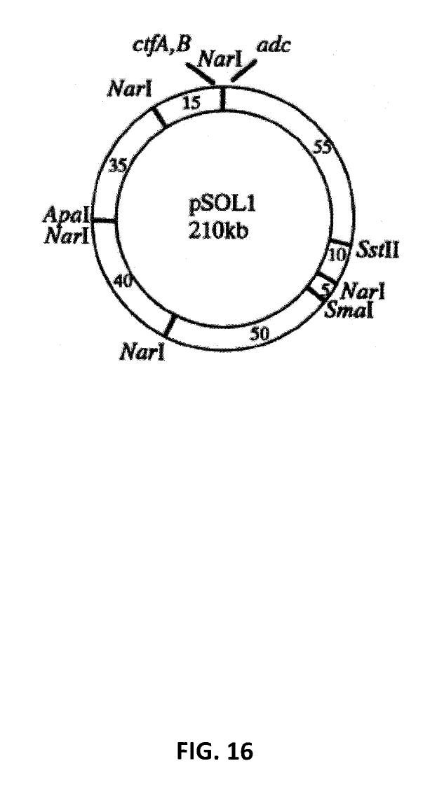

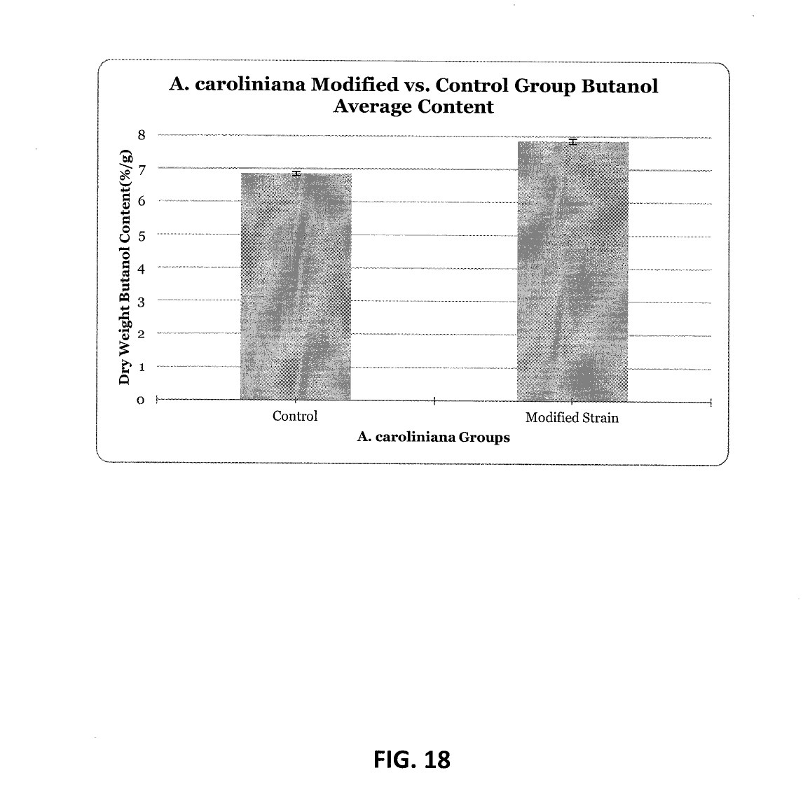

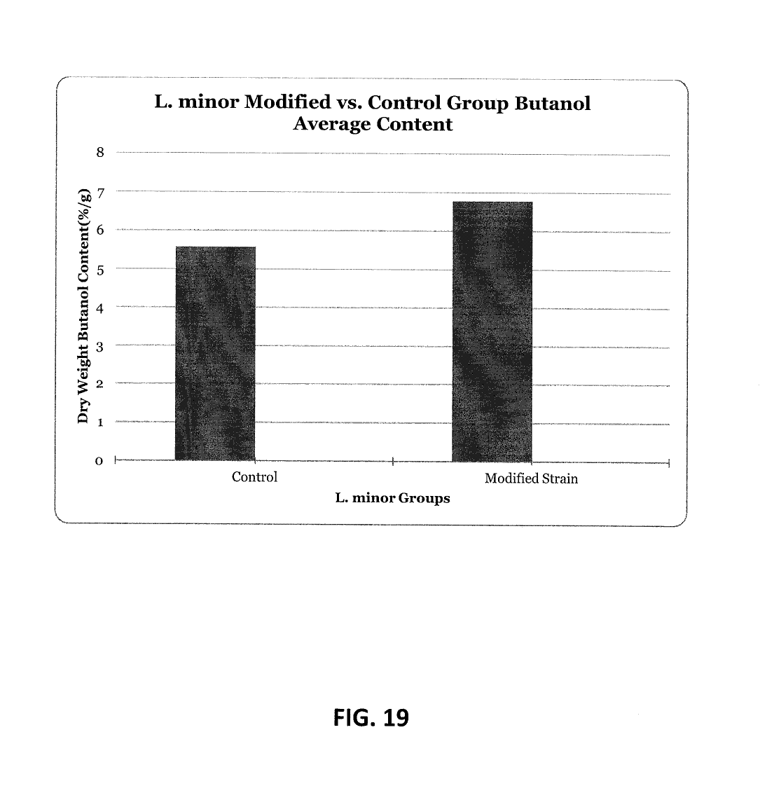

Each of FIG. 1 to FIG. 20 is described in detail in the Examples below.

### Example 1: Materials

The following list of materials was used in the disclosed methods.

Lemna minor culture, Azolla caroliniana culture

Clostridium Acetobutylicum strain, McCoy strain (ATCC 824D-5)

Watertight basin or bins capable of holding several liters of liquid

Deionized (di) water

Common laboratory supplies including incubators, shakers, centrifuges, and pH testing materials

K₂O and NO₃ fertilizer

Lighting equipment

Trypticase Soy Agar plates with 5% Defibrinated Sheep's Blood

Tomato Yellow Leaf Curl Virus—Israel Strain (IL)

BlueScript KS(+) and Drive Plasmids

Soxhlet Extractor and extraction filters and chemicals

Standard gel electrophoresis, PCR materials, and AatII/EcoRI/restriction digest materials

### Example 2: Cultivation of Unmodified L. Minor

Cultivation of unmodified L. minor occurs in a basin or bin containing only L. minor. Due to the nature of L. minor's proliferation, communal growth is a necessity. One to twelve centimeters of deionized or distilled water, maintained at a pH of 5.5 to 7.5 via primarily common peat, stands in a basin. No aerator or pump was needed. K2O and NO3 fertilizer was applied at a rate of 100 milliliters per 100 liters, or 0.001% of the volume of the solution to increase the Potassium concentration by 5 mmol. Reapplication occurs every sixteen to thirty days; depending upon the growth rate of duckweed and depletion of fertilizer; L. minor may simply be skimmed from the surface of the water and placed into a new basin containing adequate fertilizer content. Skimming and inoculation of new basins is suggested once the duckweed covers more than 85% confluency of the (surface of the) basin for maximum growth; however uniform death does not occur in overcrowded conditions, growth merely halts (though excessive amounts of fertilizer is consumed). Pasteurized cow dung was administered in doses of 1 gram per liter to supply nitrogen and phosphorus upon initial inoculation of basins, reapplication is unnecessary. The temperature is maintained at 23 degrees Celsius via supplemental heating (both surface and air). For the mother (P generation) unmodified culture, as for the cultures post-transgenesis (F generation) no photoperiod was employed; light is constant, with one 400 watt metal halide bulb per meter squared, approximately, and several fluorescent bulbs serving to amplify the light (though such intense light is certainly not necessary, it accelerates growth). Upon subsequent rapid growth, duckweed may be separated into flasks or new basins. Under such conditions, L. minor does not (and did not within in the confines of the described EXAMPLES) reproduce sexually (generally). Genetic identicality was ensured via gel electrophoresis, and samples were selected for placement into fresh basins for growth analysis. Healthy L. minor was significantly more compact, was lighter in coloration, and did not possess long roots (a sign of nutrient deprivation). Growth analysis calculated dry weight of duckweed routinely, and uniformity in growth rate was ensured.

### Example 3: Cultivation of Unmodified A. Caroliniana

A. caroliniana cultivation similarly occurs within the confines of a bin or basin, and is comparably adaptable in growing conditions. However, A. caroliniana is less tolerant of cold, grows at an overall slower rate, and tends to produce spores that may sexually reproduce, setting itself apart from L. minor. Because A. caroliniana grows at a nearly unmanageably rapid rate, communal growth for unmodified specimens is acceptable; however, because A. caroliniana produces spores, consideration and management of stock must be evaluated to ensure the purity and genetic identically of the stock. Under normal conditions, azolla tends to reproduce nearly exclusively asexually. However, because of the aforementioned, gel electrophoresis is a must to ensure identicality of the stock, and was frequently employed. Unlike duckweed, samples are preferably transferred individually to beakers and separated once budding is apparent, both because of the comparatively slower growth rate and the threat of sexual reproduction. One to twelve centimeters of deionized or distilled water, maintained at a pH of 5.5 to 7.5 via common primarily peat, stands in each container, without any aeration. As with duckweed, K2O and NO3 fertilizer is applied at a rate of 100 milliliters per 100 liters, or 0.001% of the volume of the solution to increase the Potassium concentration by 5 mmol. Pasteurized cow dung is administered in doses of 1 gram per liter to supply nitrogen and phosphorus upon initial creation of growth solution. Reapplication occurs every sixteen to thirty days; depending upon the depletion of fertilizer. Frequent inoculation of new beakers with newly split buds and undepleted growth solution solves any issues with inoculation. A. caroliniana temperature was maintained at 23 degrees Celsius via supplemental heating. As with duckweed, for the mother, unmodified culture, as for the cultures post-IL60, no photoperiod was employed and light was constant, with one 400 watt metal halide bulb per meter squared, approximately, and several fluorescent bulbs serving to amplify the light (though such intense light is certainly not necessary, it accelerates growth). Growth analysis calculated dry weight of azolla routinely, and uniformity in growth rate was ensured. A. caroliniana, when healthy, has a reddish coloration and clearly defined frond growth, which is centered upon a small crown where small roots protrude.

### Example 4: Cultivation of Clostridium Acetobutylicum

Culturing of C. Acetobutylicum was done on trypticase soy agar (TSA) plates with 5% defibrinated sheep's blood, 100 mm×15 mm size plates under standard 37° Celsius temperature incubator level. Transfer of culture tube source was done under sterile, anaerobic conditions. The anaerobic chamber in this case was a modified, airtight plastic container with pump system moving a specified gas mixture inward to create optimal environment for C. Acetobutylicum. The chamber's gas mixture consisted of 80%—N2, 10%—CO2, 10%—H2 to be pumped into the chamber as specified by the ATCC 824 manual. After anaerobic conditions were set up, transfer began by separating pellet from containment unit into 5-6 mL of Modified Reinforced Clostridial Agar/Broth (pre-reduced) to rehydrate pellet, from there 0.5 mL of the broth was used to inoculate plates which were then placed into incubator with one plate being incubated aerobically to check for contamination.

### Example 5: C. Acetobutlyicum Plasmid Isolation

Isolation of plasmid from pSol1 involved procedural extraction of plasmid via a modified procedure of a known extraction method of a similar strain of Clostridium with alterations made to fit extraction of the desired plasmid in Clostridium acetobutylicum. C. acetobutylicum cells were isolated from a main culture into a smaller subculture with a quantity of 10 ml, the cells were then treated with a 5.125 ml combined mixture of TSE buffer (Tris/Sucrose/EDTA) and lysozyme. The cells post-treatment are then treated further with 0.5 M EDTA and NaC₁₂H₂₅SO₄ solution in a process of alkaline lysis, with addition of 95% Ethanol for precipitation of DNA. The mixture was then centrifuged at high speed for 5 minutes 4° C., and further resuspended in 400 μl Tris-EDTA-Sucrose buffer at 37° C., and then another 100 μl of lysozyme solution was added and the mixture was put in an incubator for 5-10 minutes at 37° C. A further mixture of 3 N NaOH is added with another 100 mL of 0.25 EDTA-50 mM Tris. Another dosage of 100 μl of 2.0 M Tris-Hydrochloride (Neutral pH) is added as a pH buffer and mixed again via inversion, then left to stand for 3-5 minutes. A 75 μl dosage of 5.0 NaCl is added into the mixture and further left to settle for 1.5-2 hours. The mixture was centrifuged for a last time at 5 minutes and the pellet was disposed of, the leftover supernatant was treated with RNAse of pancreatic origin in a dosage of 2 μl. The final steps are adding 600 μl of phenol with a saturation of 3% NaCl plus 100 μl of chloroform-isoamyl alcohol, and then vortexing, then precipitation plus isopropanol at an equal volume at −70° C. for 10 minutes after vortexing and removal of aqueous phases. The supernatant is discarded, and tube is flipped to drain and allow DNA Pellet to drain; the very final step is to suspend the pellet in 20 μl of TE buffer gel electrophoresis.

### Example 6: IL-60-BS Construct Creation

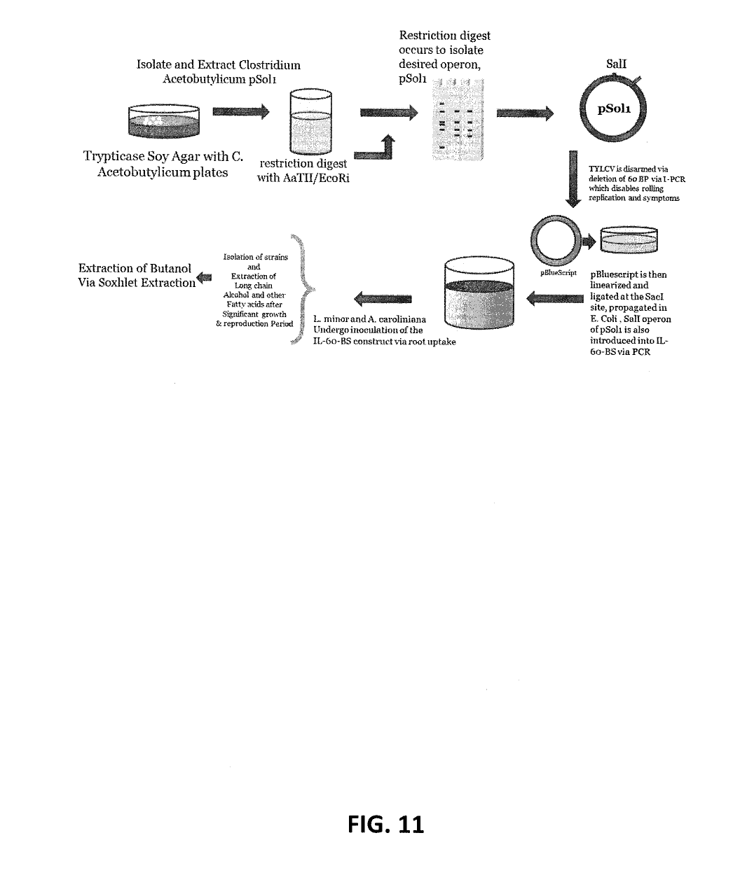

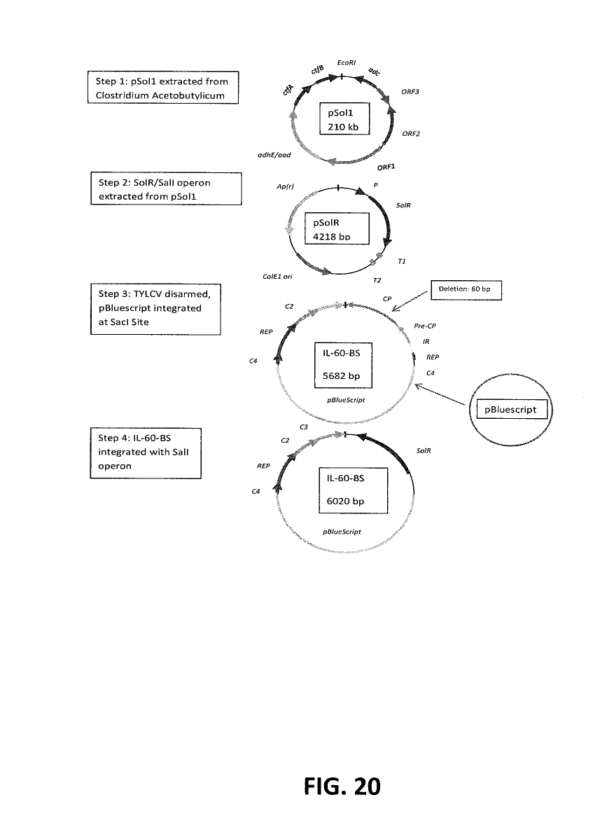

FIG. 20 shows the construction of IL-60-BS. In order to have a proper viral vector of operon expression in which the viral vector was a known crop pest, the Tomato Yellow Leaf Curl Virus, which was disarmed via deletion of 60 base pairs through Inverse-Polymerase Chain Reaction using identified primers directed outward from the ends of the deleted segment, as described in, Peretz, Yuval et al. “A Universal Expression/Silencing Vector in Plants.” Plant Physiology 145.4 (2007): 1251-1263. PMC. Web. 13 Feb. 2017. The construct is then known as the IL-60-BS but not completely modified for gene expression intentions. The next step of TYLCV modification is fusing of pBlueScript or pDrive plasmid, which is done via restriction digest to linearize and also ligate the pBlueScript or pDrive to the SacI site of the IL-60 construct. This now fully modified IL-60-BS construct of TYLCV is to be propagated into E. Coli and to have a plasmid mini-prep performed on in order to cultivate and harvest more of the construct for liquid or aqueous solution based inoculation. The fully modified IL-60-BS construct is used to inoculate the organisms described herein. The fully modified IL-60-BS construct is described in FIG. 11 and shown in FIG. 20.

### Example 7: Inoculation Process

Inoculation of A. caroliniana and L. minor is on an individual plant basis per amount of DNA needed, via an aqueous solution (as described in Gover, Ofer et al. “Only Minimal Regions of Tomato Yellow Leaf Curl Virus (TYLCV) Are Required for Replication, Expression and Movement.” Archives of Virology 159.9 2014.) with 1 μg of DNA per plant that was measured and partitioned into the solution based on the amount of plants desired to be modified. Partitioning of plants is to be done prior to exposure to inoculation solution, L. minor and A. caroliniana were inoculated in separate solutions in previously sterilized glassware. Inoculation solution was then placed under normal photoperiodic conditions and monitored over an interval.

The methods disclosed herein are different than methods previously described in the literature. The methods described herein are different based on the inoculation medium used. In addition, the methods described herein were developed and modified specifically for IL-60 inoculation in Duckweeds. In addition, the method of root trimming that was used in the disclosed methods is tailored for Duckweeds. In other words, this very specific root uptake inoculation has never been performed on Duckweeds.

### Example 8: Root-Based Inoculation Process

Materials Needed:

0.1% Tween 20

2% Gamborg's B5 medium

5˜9% Viral Load

1.5% sucrose

0.5% Jasmonic Acid

Procedure:

In deionized water, thoroughly mix 0.1% Tween 20, 2% Gamborg's B5 medium that is thoroughly agitated until well mixed.

For 96 hours, place the initial aqueous solution in a shaker at 22 degree Celsius. To better integrate components, bath sonication may be utilized.

After initial mixing process, disperse in 1.5% sucrose, and 0.5% Jasmonate at 22 degrees Celsius to create an initial aqueous solution.

Apply a viral load that is sufficient. For aggressive viruses, apply 5-9% viral load in order to induce conjugation into mixture. In order to perform targeted conjugation of a gene of interest, do so with (disarmed) viral constructs that specifically contain a gene of interest.

### Example 9: Expression of Operons

Upon inoculation of duckweeds, the initially inoculated organisms were monitored and held in isolation. A stationary camera was used to determine the P generation from subsequent generations produced via vegetative division every 12-16 hours. Operon expression and degradation was implicitly measured via analysis of elevation, or lack thereof, of butanol production from the base level.

### Example 10: Soxhlet Extraction

Each individual ‘sample’ consisted of a dried individual plant, which was weighed in grams of individually cultured plant matter and made to undergo soxhlet extraction. The measurement of butanol concentration, as such, was based upon composition of samples before and after extraction via dry weight and samples were dried on a drying rack or inside an autoclave on the drying ex+ for 4-7 hours. Ethanol was used as the extraction chemical of choice for both L. Minor and Azolla. Soxhlet extraction methodology varies between needed samples, but both A. caroliniana and L. Minor were both able to be extracted via the ethanol/methanol solution. Extraction of butanol via soxhlet extraction is described in FIG. 11 and FIG. 12.

### Example 11: Results for A. Caroliniana and L. Minor

Select samples of both A. caroliniana and L. minor had a P generation butanol elevation of over 3% in terms of percent efficiency after operon expression. The T-test statistical test for L. minor proved that the standard deviations of the data set are in fact statistically significant within the P generation; the A. caroliniana data set yielded similar results in statistical significance, albeit to a lesser extent. The subsequent generations showed retention of expressed operons although actively showed degradation; no reliably significant elevation was witnessed after the F₃ generation for A. caroliniana and the F₄ generation for L. minor, illustrated in the respective Figures.

“Percent Efficiency” refers to production efficiency, or dry weight butanol percentage. The term “percent efficiency” is used, as such, to model the elevation of butanol, which occurs naturally in both A. caroliniana and L. minor.

TABLE 1
AZOLLA CAROLINIANA MODIFICATION RESULTS

| Successfully Modified Azolla C. | Dry Weight Percentage |
|---|---|
| Samples and generation | Efficiency per Gram |
| A-1-Control Sample 1 | 7.45 |
| A-2-Control Sample 2 | 8.11 |
| A-3-Control Sample 3 | 5.71 |
| A-4-Control Sample 4 | 5.78 |
| A-5-Control Sample 5 | 7.18 |
| A-6-Strain AA P | 9.2 |
| A-7-Strain AA F1 | 8.47 |
| A-8-Strain AA F2 | 7.58 |
| A-9-Strain AA F3 | 7.11 |
| A-10-Strain AA F4 | 7.08 |
| A-11-Strain AB P | 8.13 |
| A-12-Strain A F1 | 7.56 |
| A-13-Strain AB F2 | 7.14 |
| A-14-Strain AB F3 | 7.23 |
| A-15-Strain AB F4 | 7.16 |
| A-16-Strain AC P | 9.89 |
| A-17-Strain AC F1 | 8.14 |
| A-18-Strain AC F2 | 6.57 |
| A-19-Strain AC F3 | 8.17 |
| A-20-Strain AC F4 | 6.94 |

TABLE 2
STATISTICAL ANALYSIS OF MODIFIED
STRAINS OF AZOLLA CAROLINIANA

| t-Test: Paired Two Sample | | |
|---|---|---|
| for Means | Variable 1 | Variable 2 |
| Mean | 6.846 | 7.748 |
| Variance | 0.9644828571 | 0.8251742857 |
| Observations | 15 | 15 |
| Pearson Correlation | 0.5969115723 | |
| Hypothesized Mean | | |
| Difference | 0.01 | |
| df | 14 | |
| t Stat | −4.149367345 | |
| P(T <= t) one-tail | 0.0004913888328 | |
| t Critical one-tail | 1.761310115 | |
| P(T <= t) two-tail | 0.0009827776656 | |
| t Critical two-tail | 2.144786681 | |

TABLE 3
LEMNA MINOR MODIFICATION RESULTS

| | |
|---|---|
| D-1 Control | 5.12 |
| D-2 Control | 5.82 |
| D-3 Control | 6.47 |
| D-4 Control | 3.12 |
| D-5 Control | 7.12 |
| D-6 Control | 5.25 |
| D-7 Control | 6.09 |
| DA-1 P | 8.41 |
| DA-1 F1 | 7.07 |
| DA-1 F2 | 6.02 |
| DA-1 F3 | 6.1 |
| DA-1 F4 | 5.78 |
| DA-1 F5 | 6.3 |
| DA-1 F6 | 6.02 |
| DB-2 P | 6.05 |
| DB-2 F1 | 5.8 |
| DB-2 F2 | 6.2 |
| DB-2 F3 | 5.4 |
| DB-2 F4 | 6.09 |
| DB-2 F5 | 5.06 |
| DB-2 F6 | 6.45 |
| DC-3 P | 9.89 |
| DC-3 F1 | 8.71 |
| DC-3 F2 | 6.98 |
| DC-3 F3 | 6.87 |
| DC-3 F4 | 6.11 |
| DC-3 F5 | 6.24 |
| DD-4 P | 8.81 |
| DD-4 F1 | 8.2 |
| DD-4 F2 | 7.01 |
| DD-4 F3 | 6.9 |
| DD-4 F4 | 6.54 |
| DD-4 F5 | 6.6 |
| DD-4 F6 | 6.02 |
| DE-5 P | 8.71 |
| DE-5 F1 | 7.45 |
| DE-5 F2 | 7.34 |
| DE-5 F3 | 6.02 |
| DE-5 F4 | 7.12 |

TABLE 4
STATISTICAL ANALYSIS OF MODIFIED
STRAINS OF LEMNA MINOR

| t-Test: Paired Two | | |
|---|---|---|
| Sample for Means | Variable 1 | Variable 2 |
| Mean | 5.551538462 | 6.768461538 |
| Variance | 1.53842915 | 1.176018623 |
| Observations | 39 | 39 |
| Pearson | | |
| Correlation | −0.03294690507 | |
| Hypothesized Mean | | |
| Difference | 1 | |
| df | 38 | |
| t Stat | −8.269232449 | |
| P(T <= t) one-tail | 0.0000000002534031569 | |
| t Critical one-tail | 1.685954405 | |
| P(T <= t) two-tail | 0.0000000005068063139 | |
| t Critical two-tail | 2.024394147 | |

Hypothesized content refers to Butanol, obtained from the null (unexpressed) control via soxhlet extraction. All samples were dried before weighing. The percent is, as such, the percentage of butanol content per dry weight.

### Example 12: Growth Analyses for A. Caroliniana and L. Minor

The growth rate, over a five-month period of time, of the macroalgal duckweed Azolla Caroliniana and Lemna Minor, demonstrated as doubling time, or a doubling of dry weight biomass, was unimpaired by modification procedure; our data resulted in statistically insignificant grown differences between the samples that consisted of both modified and unmodified organisms. The rate of full potential growth in Azolla caroliniana did not show significant shift in comparison between the unmodified and modified strains, and the known doubling rate of Azolla biomass (dry weight). Prior research suggested a doubling time of about 6.1 days in Azolla sp. while L. Minor had a known rate of about 16-36 hours.

TABLE 5
GROWTH RATES

| | Averaged Doubling |
|---|---|
| Strains: | Time(Hours): |
| Unmodified Azolla caroliniana (Control) | 152.1 |
| Unmodified Lemna Minor (Control) | 19.7 |
| Modified Azolla Caroliniana Strains: | |
| AA | 145.5 |
| AB | 161.8 |
| AC | 157.1 |
| L. Minor Strains | |
| DA | 17.4 |
| DB | 21.1 |
| DC | 23.9 |
| DD | 15.4 |
| DE | 18.5 |

The conditions of all strain-samples were regulated and standardized in collection of the data via weighing of dry samples (achieved via insertion onto the drying rack of an autoclave on the drying ex+ for 4-7 hours) after cultivation. The data clearly shows that the Azolla caroliniana and Lemna minor averages are consistent in showing that the growth times of the modified and unmodified strains were functionally identical, leading us to conclude that the genetic expression of operons via IL-60 constructs did not in fact affect how the rate of growth per sample on a statistically noticeable level.

### Example 13: Alternative Plant Hosts

In the future, IL-60 based elevation of butanol may be applied to larger plants that are already utilized for biofuel commercially, such as canola, as the nontransgenic expression of biofuel elevating traits modeled by the disclosed research may increase production via simple injection.

### DISCUSSION

Overall, samples of both A. caroliniana and L. minor had a P generation butanol elevation of over 3% in terms of percent efficiency after operon expression. The T-test for L. minor proved that the standard deviations of the data set are in fact statistically significant within the P generation; the A. caroliniana data set yielded similar results in statistical significance, albeit to a lesser extent; both were confirmed to be statistically significant in elevation not only in the P generation but also in subsequent generations via statistical tests. The subsequent generations showed retention of expressed operons although actively showed degradation; no reliably significant elevation was witnessed after the F3 generation for A. caroliniana and the F4 generation for L. minor. In addition, L. minor grows at a rate of up to 8.3 times that of Azolla sp.

However, both Azolla caroliniana and Lemna minor, in addition to other macroalgal ferns can be used for the purpose of sustainable, carbon negative biomass and biofuel production through the use of IL60 mediated gene expression. The pSol1 plasmid's known association with elevation of butanol production was further confirmed to remain valid even when expressed via a viral construct in eukaryotic organisms; in the P generation immediately following expression the expression was most effective, while in subsequent generations active degradation was witnessed until a regression to base butanol levels by the F3 generation in Azolla and the F4 generation in duckweed was witnessed; it is inferred that as generations subsequently continued expression, the expression via the viral construct decreased sharply, with a near exponential drop after the first reproduction cycle (P to F1) in nearly all circumstances. Thus, duckweeds serve as model organisms that relay the rate of decadence transgenerationally, in regards to expression of genes that, through IL-60's known quality as a universal (for vascular plants) vector, may be directly projected onto current commercial biofuel producing organisms, including canola and corn. The shown success of expression means that further expression via IL-60 construct may allow for inducible gene expression for the first time in adult plants, versus conventional transgenic methods which traditionally require embryonic states to facilitate expression.

IL-60 based expression of the pSol1 plasmid's operons induced statistically significant elevation as compared to naturally occurring butanol levels, and subsequently modeled transgenerational decay of IL-60 based operon expression in A. caroliniana and L. minor. Based upon the above-described experiments, A. caroliniana and L. minor, are confirmed to be useful organisms (hosts) for elevated butanol production, resulting in renewable, reliable, carbon negative biogasoline production from fast growing macroalgal sources.

While certain embodiments have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed:

1. A modified macroalga duckweed useful for production of acetone, butanol, or ethanol.

2. The modified microalga duckweed of claim 1, wherein the duckweed is Azolla caroliniana or Lemna minor.

3. The modified macroalga duckweed of claim 2, wherein the modification is inoculation with a viral-based construct.

4. The modified macroalga duckweed of claim 3, wherein the viral-based construct is IL-60-BlueScript (BS).

5. The modified macroalga duckweed of claim 3, wherein the inoculation is via root uptake in the macroalga duckweed.

6. The modified macroalga duckweed of claim 3, wherein the viral-based construct comprises a 60-base-pair deletion inhibiting rolling circle replication.

7. The modified macroalga duckweed of claim 3, wherein the viral-based construct comprises ctfA, ctfB, adc, and aad.

8. The modified macroalga duckweed of claim 1, wherein the modified macroalga duckweed has an increase in dry weight acetone content (%/g), dry weight butanol content (%/g), or dry weight ethanol content (%/g), as compared to an unmodified (control) macroalga duckweed of the same species.

9. The modified macroalga duckweed of claim 8, wherein the increase in dry weight acetone content (%/g), dry weight butanol content (%/g), or dry weight ethanol content (%/g), is 0.01% to 0.5%, 0.5% to 1%, 1% to 1.5%, 1.5% to 2%, 2% to 2.5%, 2.5% to 3%, 3% to 3.5%, or 3.5% or more.

10. The modified macroalga duckweed of claim 1, wherein the modified macroalga duckweed has an increase in dry weight acetone content (%/g), dry weight butanol content (%/g), or dry weight ethanol content (%/g), as compared to an unmodified (control) macroalga duckweed of the same species over a period of time.

11. The modified macroalga duckweed of claim 10, wherein the period time is a generation or more.

12. The modified macroalga duckweed of claim 1, wherein the modified macroalga duckweed has a similar growth rate as compared to an unmodified (control) macroalga duckweed of the same species over a period of time.

13. The Modified it alga duckweed of claim 12, wherein the period of time is a generation or more.

14. The modified macroalga duckweed of claim 3, wherein the viral-based construct comprises a disarmed tomato yellow leaf curl virus (TYLCV) and a 210 kb plasmid (pSOL1) obtained from Clostridium acetobutylicum ATCC 824 comprising, ctfA, ctfB, adc, and aad.

15. The modified macroalga duckweed of claim 3, wherein IL-60-BS comprises a disarmed tomato yellow leaf curl virus (TYLCV) and a SolR/SalI operon of pSOL1.

16. A modified Azolla caroliniana wherein the modification is the induction of the Azolla caroliniana with a viral-based vector comprising a disarmed tomato yellow leaf curl virus (TYLCV) and a 210 kb plasmid (pSOL1) obtained from Clostridium acetobutylicum ATCC 824 comprising ctfA, ctfB, adc, and aad, wherein the induction is via root uptake and wherein ctfA, ctfB, adc, and aad are expressed and expression results in increased production of acetone or butanol as compared to an unmodified Azolla caroliniana.

17. A modified Lemna minor wherein the modification is the induction of the Lemna minor With a viral-based vector comprising a disarmed tomato yellow leaf curl virus (TYLCV) and a 210 kb plasmid (pSOL1) obtained from Clostridium acetobutylicum ATCC 824 comprising ctfA, ctfB, adc, and aad, wherein the induction is via root uptake and wherein ctfA, ctfB, adc, and aad are expressed and expression results in increased production of acetone or butanol as compared to an unmodified Lemna minor.
